(19) United States
(12) Patent Application Publication
(10) Pub. No.: US 2016/0152538 A1
(43) Pub. Date: Jun. 2, 2016

(54) REPELLENT EFFECTIVE AGAINST ANOPHELES GAMBIAE

(71) Applicant: Simon Fraser University, Burnaby (CA)

(72) Inventors: Erika Plettner, Burnaby (CA); Bernard Roitberg, North Vancouver (CA)

(73) Assignee: Simon Fraser University, Burnaby (CA)

(21) Appl. No.: 14/956,041

(22) Filed: Dec. 1, 2015

Related U.S. Application Data
(60) Provisional application No. 62/086,058, filed on Dec. 1, 2014.

(51) Int. Cl. C07C 43/215; A01N 31/16; C07C 43/205

(52) U.S. Cl. CPC C07C 43/215; C07C 43/2055; A01N 31/16

(57) ABSTRACT

The present disclosure describes methods for repelling an Anopheles gambiae including applying to a site of interest an effective amount of a compound of Formula I:

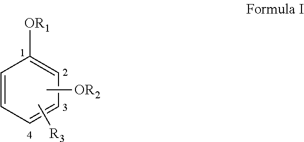

wherein:
R₁ is methyl, ethyl, propyl, n-butyl, or allyl;
R₂ is at positions 2, 3 or 4 and is H, methyl, ethyl, propyl, n-butyl, or allyl; and
R₃ is optionally present at positions 2, 3 and 4, and is allyl;
   with the provisos that
when R₂ is at position 2, R₃ if present is at position 3, or
when R₂ is at position 3, R₃ if present is at positions 2 or 4, or
when R₂ is at position 4, R₃ if present is at position 2; and
   with the proviso that the compound of Formula I does not include a compound according to Formula II:

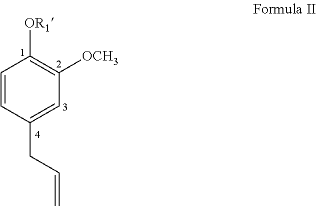

wherein R₁′ is methyl, ethyl, propyl, n-butyl, or allyl;
or mixture thereof to a site of interest.

### CROSS-REFERENCE TO RELATED APPLICATION

This application claims the benefit of U.S. Patent Application No. 62/086,058, filed Dec. 1, 2014, the disclosure of which is hereby incorporated by reference in its entirety.

### BACKGROUND

Malaria is a major health concern affecting 216 million people worldwide in 2010. The causative agent of malaria is the parasite Plasmodium, which is transmitted to humans during bloodfeeding by Anopheles mosquitoes. Control strategies to limit the impact of this disease focuses broadly on two main approaches: prevention and case management. Prevention focuses mainly on insect vector control as a means of reducing malaria incidence. The aim of vector control is to reduce the number of Anopheles mosquito attacks on humans in endemic areas, thereby reducing transmission of Plasmodium parasites to humans. This is done mainly through the use of insecticide-treated bednets (“ITNs”) and indoor residual spraying (“IRS”). Although these strategies have been effective, they have led to a change in Anopheles mosquito behaviour such that females, who previously only attacked humans at night, are now attacking earlier in the day, before humans are under bednets. This has led to a need for protective repellents in addition to other control measures in order to reduce the attack rate of Anopheles mosquitoes before nightfall.

DEET™ (N—N-diethyl-meta-toluamide) is a widely used repellent that is effective against many insects including Anopheles mosquitoes. However, there is concern about adverse health effects from the use of DEET™, especially in children and at high concentrations. The basis for these adverse effects may be the inhibition of acetylcholinesterase and butylcholinesterase by DEET™ at concentrations typically found in repellent products. Because of this, there is a need to develop effective mosquito repellents that can reduce these adverse effects.

Several studies have shown that DEET™ interacts with the olfactory system of insects it repels. For example, Culex quinquefasciatus and Aedes aegypti mosquitoes have been shown to detect DEET™ directly. Additionally, it has been shown that DEET™ inhibits olfactory receptor neurons on the maxillary palps of A. gambiae and thereby decreases the response of these neurons to 1-octen-3-ol, an attractant produced by humans. Interestingly, in C. quinquefasciatus and A. aegypti this inhibition was not detected. DEET™ has also been shown to stimulate neurons in the sacculus on the antenna of Drosophila melanogaster that express an ionotropic receptor (IR40a). When IR40a expression is abolished, repellence of D. melanogaster to DEET™ disappears.

Accordingly, there is a need for new repellents that are effective against Anopheles mosquitoes that has arisen due to changes in vector behaviour and as a result of control strategies and concern over the health impacts of current repellents. The repellent should cause mosquitoes to reduce their contact with a blood-host odor and probe less at the host odor. The repellent should also be non-toxic to the host (e.g., humans).

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure presents methods for repelling of an Anopheles gambiae including applying to a site of interest an effective amount of a compound of Formula I:

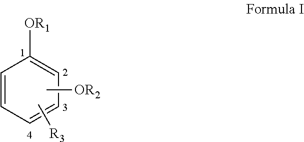

wherein:

R₁ is methyl, ethyl, propyl, n-butyl, or allyl;

R₂ is at positions 2, 3 or 4 and is H, methyl, ethyl, propyl, n-butyl, or allyl; and

R₃ is optionally present at positions 2, 3 and 4, and is allyl;

with the provisos that

when R₂ is at position 2, R₃ if present is at position 3, or

when R₂ is at position 3, R₃ if present is at positions 2 or 4, or

when R₂ is at position 4, R₃ if present is at position 2; and

with the proviso that the compound of Formula I does not include a compound according to Formula II:

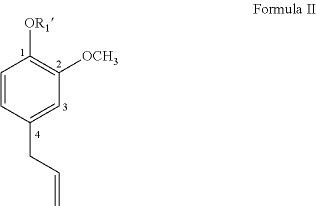

wherein R₁′ is methyl, ethyl, propyl, n-butyl, allyl, or mixture thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an illustration of the structures of embodiments of compounds tested in electroantennogram (EAG) assays as potential repellents for A. gambiae. Names given to compounds reflect the chemical structure of the compounds.

FIG. 2 is a schematic representation of an olfactometer arena. a: release chamber. b: experiment arena. c: human odor/chemical complex. d: plant odor source. e: air pump. f: release gate. The arrow labeled CO₂ indicates the location of the tube the experimenter breathed in during trials.

FIG. 3 is a graph showing the physiological response of A. gambiae to potential repellents and a human odor measured through EAG screening. Activity indicates the response of A. gambiae to the repellent/human odor source combination compared to a DEET™ reference peak. Chemical concentrations tested (1 μg, 10 μg, and 100 μg) are pooled, as chemical concentration did not affect A. gambiae response.

FIG. 4 is a graph showing the air-corrected antennal depolarizations (mV) of A. gambiae in response to potentially repellent compounds.

FIGS. 5A-5C are graphs showing the behavioural response of A. gambiae to a human odor protected with potentially repellent compounds. Solvent types are pooled, as this was not found to affect behaviour of A. gambiae. FIG. 5A: Total experiment time (sec) A. gambiae persisted in the experiment arena. FIG. 5B: Amount of time (sec) A. gambiae landed on the human odor source during trials. FIG. 5C: Number of times A. gambiae attempted to probe at the human odor source during trials. Error bars indicate 95% confidence intervals. Chemical treatments that differ significantly from each other through Tukey's HSD tests are indicated with different letters.

FIG. 6 is a graph showing depolarization responses of A. gambiae to embodiments of compounds of the present disclosure by themselves (100 μg dose) and a stocking stimulus by itself in the following stimulation sequence of female mosquito antennae: Air 1, Air 2, DEET1, compound, Air 3, Air 4, and stocking. The “Air” stimuli are blanks prepared by treating a stimulus-holding paper disk with hexane and allowing the hexane to evaporate fully prior to using the stimulus. Responses shown in the graph corrected responses obtained with the compound stimulus or the stocking stimulus with the larger of the two preceding air stimuli. Bars represent mean+/−S.E. of 10 replicates.

### DETAILED DESCRIPTION

The present disclosure describes methods for repelling of an Anopheles gambiae including applying to a site of interest an effective amount of a compound of Formula I:

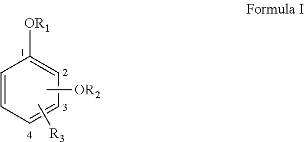

wherein:

R₁ is methyl, ethyl, propyl, n-butyl, or allyl;

R₂ is at positions 2, 3 or 4 and is H, methyl, ethyl, propyl, n-butyl, or allyl; and

R₃ is optionally present at positions 2, 3 and 4, and is allyl,

with the provisos that

when R₂ is at position 2, R₃ if present is at position 3, or

when R₂ is at position 3, R₃ if present is at positions 2 or 4, or

when R₂ is at position 4, R₃ if present is at position 2; and

with the proviso that the compound of Formula I does not include a compound according to Formula II:

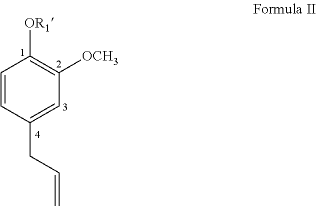

wherein R₁′ is methyl, ethyl, propyl, n-butyl, allyl, or mixture thereof.

In some embodiments, the compound of Formula I decreases arthropod (e.g., Anopheles gambiae) attacks on humans.

In some embodiments, the methods include applying to a site of interest an effective amount of a compound of Formula I:

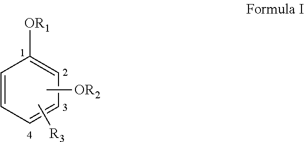

wherein:

R₁ is methyl, ethyl, propyl, n-butyl, or allyl,

R₂ is at positions 2, 3 or 4 and is ethyl, propyl, n-butyl, or allyl, and

R₃ is optionally present at positions 2, 3 and 4, and is allyl,

with the provisos that

when R₂ is at position 2, R₃ if present is at position 3, or

when R₂ is at position 3, R₃ if present is at positions 2 or 4, or

when R₂ is at position 4, R₃ if present is at position 2; and

with the proviso that the compound of Formula I does not include a compound according to Formula II:

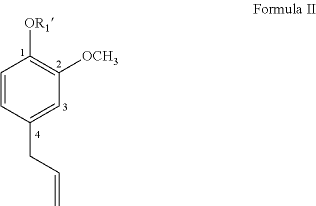

wherein R₁′ is methyl, ethyl, propyl, n-butyl, allyl, or mixture thereof.

In some embodiments, the methods include applying to a site of interest an effective amount of a compound of Formula I:

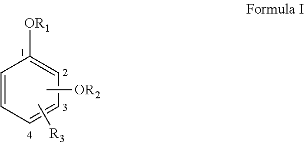

wherein:

R₁ is methyl, ethyl, propyl, n-butyl, or allyl;

R₂ is at positions 2, 3 or 4 and is ethyl, propyl, n-butyl, or allyl; and

R₃ is absent.

In some embodiments, the compound of Formula I is

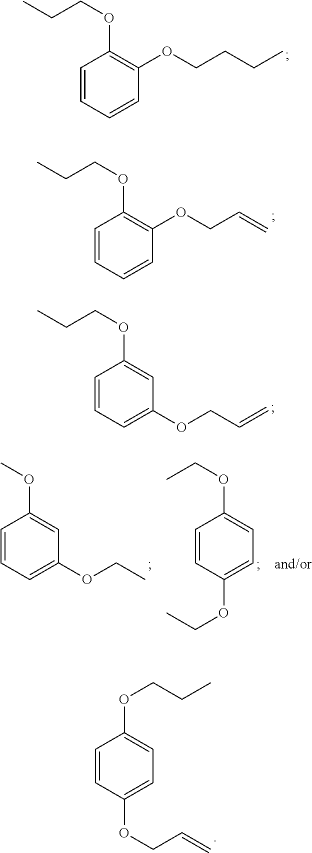

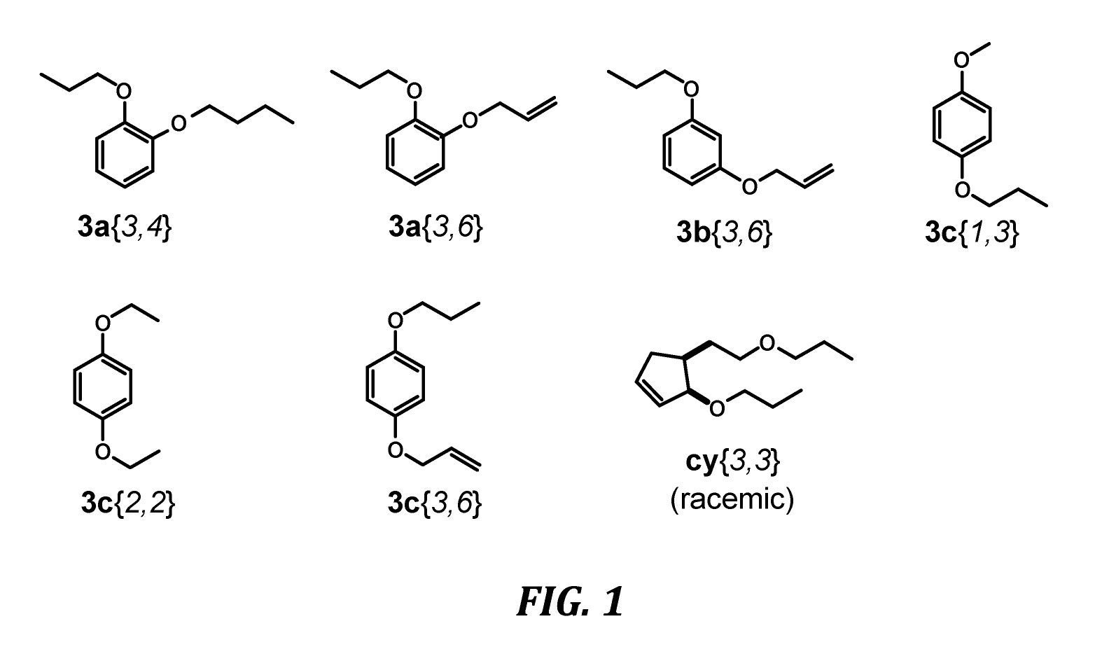

In some embodiments, the compound of the present disclosure is a repellent for Anopheles gambiae. As used herein, “compound of the present disclosure” or “compound of Formula I” includes a single compound of Formula I or a mixture of compounds of Formula I. The compound of the present disclosure can be used to repel an Anopheles gambiae arthropod attack on a human, such that an Anopheles gambiae is deterred from probing at the human blood host. In some embodiments, the compound of Formula (I) is 1-allyloxy-4-propoxybenzene (3c{3,6}), as shown in FIG. 1.

Without wishing to be bound by theory, it is believed that Anopheles gambiae, in the absence of repellents, exhibits probing at a blood host or a simulated blood host. In some embodiments, the compound of the present disclosure can reduce probing at a blood host or a simulated blood-host and/or can increase a probability that an Anopheles gambiae seeks a plant odor source instead of the blood host or the simulated blood host.

In some embodiments, the sites of interest to which the compound of the present disclosure is applied include bed nets and/or skin (e.g., a skin portion). The compound of the present disclosure can be applied in an amount of 1 μg/cm² or more (e.g., 5 μg/cm² or more, 10 μg/cm² or more, or 50 μg/cm² or more) and/or 100 μg/cm² or less (e.g., 50 μg/cm² or less, 10 μg/cm² or less, or 5 μg/cm² or less).

In some embodiments, the compound of the present disclosure regulates a behavioural response (e.g., a physiological olfactory response) of Anopheles gambiae, such the choice of a female mosquito in the presence of a compound of the present disclosure to approach a human host and to attack the human host is a modified compared a female mosquito in the absence of a compound of the present disclosure. In some embodiments, the compound of the present disclosure can reduce malaria incidence by reducing mosquito-based transmission of a Plasmodium parasite. As an example, at a 1% w/v dose on an artificial host (e.g., stocking with human odor), the compound of the present disclosure can statistically significantly decrease the number of probes the mosquitoes make at the host (i.e., from an average of 5 and a maximum of 50 to an average of 0 and a maximum of 7), and/or the compound of the present disclosure can significantly decrease the total amount of time that the mosquitoes probe at the host (from an average of 100 second and a maximum of 1400 seconds to an average of 0 seconds and a maximum of 100 seconds).

### Compositions

The compound of the present disclosure (e.g., a repellant compound) can be incorporated into a composition. The composition can include one or more compounds of the present disclosure. For example, the compound of the present disclosure can be mixed in a dermatologically acceptable-carrier. The carrier can allow the formulation to be adjusted to an effective concentration of the compound of the present disclosure. The carrier can further provide water repellency, decrease skin irritation, and/or soothe and condition skin. For example the carrier may include silicone, petrolatum, lanolin, a polymer, or many of several other well-known carrier components.

Desirable properties of a topical insect repellent include low toxicity, resistance to loss by water immersion or sweating, low or no odor or at least a pleasant odor, ease of application, and rapid formation of a dry tack-free surface film.

Examples of organic liquid carriers include liquid aliphatic hydrocarbons (e.g., pentane, hexane, heptane, nonane, decane and their analogs) and liquid aromatic hydrocarbons. Examples of other liquid hydrocarbons include oils produced by the distillation of coal and the distillation of various types and grades of petrochemical stocks, including kerosene oils which are obtained by fractional distillation of petroleum. Other petroleum oils include those generally referred to as agricultural spray oils (e.g., the so-called light and medium spray oils, consisting of middle fractions in the distillation of petroleum and which are only slightly volatile). Such oils are usually highly refined and may contain only minute amounts of unsaturated compounds. Such oils, moreover, are generally paraffin oils and accordingly can be emulsified with water and an emulsifier, diluted to lower concentrations, and used as sprays. Tall oils, obtained from sulfate digestion of wood pulp, like the paraffin oils, can similarly be used. Other organic liquid carriers can include liquid terpene hydrocarbons and terpene alcohols such as alpha-pinene, dipentene, terpineol, and the like.

Other carriers include silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials.

In addition to the above-mentioned liquid hydrocarbons, the carrier can contain conventional emulsifying agents which can be used for causing the compound of the present disclosure to be dispersed in, and diluted with, water for end-use application.

Still other liquid carriers can include organic solvents such as aliphatic and aromatic alcohols, esters, aldehydes, and ketones. Aliphatic monohydric alcohols include methyl, ethyl, normal-propyl, isopropyl, normal-butyl, sec-butyl, and tert-butyl alcohols. Suitable alcohols include glycols (such as ethylene and propylene glycol) and pinacols. Suitable polyhydroxy alcohols include glycerol, arabitol, erythritol, sorbitol, and the like. Finally, suitable cyclic alcohols include cyclopentyl and cyclohexyl alcohols.

Aromatic and aliphatic esters, and aldehydes and ketones can be used as carriers, and occasionally are used in combination with the above-mentioned alcohols. Still other liquid carriers include relatively high-boiling petroleum products such as mineral oil and higher alcohols (such as cetyl alcohol). Additionally, stabilizers (e.g., tert-butyl sulfinyl dimethyl dithiocarbonate) can be used in conjunction with or as a component of the carrier or carriers.

Solid carriers which can be used with the compound of the present disclosure include finely divided organic and inorganic solid materials. Suitable finely divided solid inorganic carriers include siliceous minerals such as synthetic and natural clay, bentonite, attapulgite, fuller's earth, diatomaceous earth, kaolin, mica, talc, finely divided quartz, and the like, as well as synthetically prepared siliceous materials, such as silica aerogels and precipitated and fume silicas. Examples of finely divided solid organic materials include cellulose, sawdust, synthetic organic polymers, and the like. Examples of semi-solid or colloidal carriers include waxy solids, gels (such as petroleum jelly), lanolin, and the like, and mixtures of well-known liquid and solid substances which can provide semi-solid carrier products.

Insect repellent compositions containing the compound of the present disclosure can contain adjuvants known in the art of personal care product formulations, such as thickeners, buffering agents, chelating agents, preservatives, fragrances, antioxidants, gelling agents, stabilizers, surfactants, emollients, coloring agents, aloe vera, waxes, other penetration enhancers and mixtures thereof, and therapeutically or cosmetically active agents.

The compositions can contain other adjuvants such as one or more therapeutically or cosmetically active ingredients. Exemplary therapeutic or cosmetically active ingredients useful in the compositions of the invention include fungicides, sunscreening agents, sunblocking agents, vitamins, tanning agents, plant extracts, anti-inflammatory agents, anti-oxidants, radical scavenging agents, retinoids, alpha-hydroxy acids, emollients, antiseptics, antibiotics, antibacterial agents or antihistamines, and may be present in an amount effective for achieving the therapeutic or cosmetic result desired.

The compound of the present disclosure can be used individually or combined in any proportion. In general, the composition of the compound of the present disclosure should contain sufficient amounts of active insect repellent compound to be efficacious in repelling the insect from the host over a prolonged period of time (preferably, for a period of at least several hours).

The amount of each repellant compound of Formula I or mixtures thereof in an insect repellent composition or repellent article can, in some embodiments, not exceed about 80% by weight based on the weight of the final product, however, greater amounts can be utilized in certain applications and this amount is not limiting. In some embodiments, a suitable amount of the repellent compound of the present disclosure can be at least 0.001% by weight (e.g., about 0.01% by weight) up to 50% by weight (e.g., about 20% by weight), based on the weight of the composition or article. Specific compositions will depend on the intended use. In some embodiments, the repellent compounds of the present disclosure can be formulated without a carrier and be effective.

The compound of the present disclosure can be formulated and packaged so as to deliver the compound in a variety of forms including as a solution, suspension, cream, ointment, gel, film or spray, depending on the preferred method of use. The carrier can be an aerosol composition adapted to disperse the compound of the present disclosure into the atmosphere by means of a compressed gas.

The composition can be used as a topical insect repellent article, such as colognes, lotions, sprays, creams, gels, ointments, bath and shower gels, foam products (e.g., shaving foams), makeup, deodorants, shampoo, hair lacquers/hair rinses, and personal soap compositions (e.g., hand soaps and bath/shower soaps), air fresheners, candles, scented articles, fibers, sheets, cloth (e.g., for clothing, nettings (mosquito netting), and other fabrics), paper, paint, ink, clay, woods, furniture (e.g., for patios and decks), carpets, sanitary goods, plastics, polymers, and the like.

Example 1 investigates A. gambiae response to potential repellents through an electroantennogram screening assay and the most promising of these candidates (1-allyloxy-4-propoxybenzene, 3c{3,6}) was chosen for behavioural testing. Example 1 also describes an assay to evaluate blood-host seeking behaviour of A. gambiae towards a simulated host protected with this repellent. The compound 3c{3,6} was found to be an effective repellent, causing mosquitoes exposed to it to reduce their contact with a blood-host odor and probe less at the host odor.

### EXAMPLE

### Example 1

### Electroantennography Study of A. gambiae Repellents

Because olfaction appears to be involved in DEET™ repellence, potentially repellent diethers were initially screened by electroantennography (EAG) to assess the physiological response of A. gambiae to these chemicals. The aim of this screen was to determine which chemicals A. gambiae was able to detect and how the physiological response produced when presented with these compounds.

In order to assess whether the physiological response observed in the EAG screening translates into a behavioural change in A. gambiae, a behavioural assay was performed to evaluate blood-seeking behaviour of A. gambiae in response to a chemically protected host in the presence of alternative foodstuffs. Here, resources that support somatic and gametic function were used to the assay's advantage. Female mosquitoes have two potential food sources: nectar from a plant source or a blood meal from a blood-host. A blood meal is riskier to obtain due to the defensive host; however, the nutrients provided are necessary for egg production, making obtaining a blood meal a necessary component to increase reproductive fitness. On the other hand, carbohydrates, primarily from plant sources are needed to support somatic function as well as fueling the search for blood hosts. In addition, there is limited space in a mosquito's crop that necessitates a choice between these two food sources for a mosquito. With these factors in mind an assay was developed to evaluate A. gambiae behaviour when odors from these two food sources were present.

One potential repellent was chosen from the EAG screen and whether mosquitoes would alter their behaviour when presented with a host odor protected with this chemical and an alternate plant odor source was assessed. The aim of the treatment was that the repellent chemical covering the potential blood-host (simulated through a human odor) would push the mosquito away from the potential blood-host. Additionally, a plant odor source would pull the mosquito towards a different food source and away from the potential blood-host. This is similar to a technique used in African agriculture to increase crop yields termed “push-pull” technology in which insect pests are diverted from crops through the use of repellent plants intercropped with the crop of interest and attractive plants planted at the fringe of crops. Similarly, in the employed behavioural assays, this chemical would cause reduced probing at the simulated blood-host (similar to that found for DEET™) and an increased likelihood of seeking the plant odor source.

### Methods and Materials

Anopheles gambiae Colony.

Anopheles gambiae was collected from Njagi, Tanzania in 1997. The colony was maintained in a temperature and humidity-controlled chamber used to maintain the mosquito colony (Conviron™) at 28° C., 80% relative humidity (RH), 12 hours dark:12 hours light (12D:12L) photoperiod and a one hour twilight transition. Adult mosquitoes were held in 30 cm³ Perspex cages with mesh on 3 sides and blood-fed weekly by placing an exposed forearm in the cage for 10-12 minutes after adults (4-7 days post-eclosion) had been starved with distilled water for 24 hours. Eggs were collected on moist filter paper within 48 hours of blood feeding. Eggs were washed with distilled water into a glass bowl and left another 48 hours, after which 200 hatched larvae were transferred into plastic trays (30 cm×45 cm×6 cm) filled with 2 cm of distilled water. Larvae were fed with flake fish food (Nutrafin Basix Staple Food) and upon pupation, were transferred in water filled dishes to colony cages with access to 5% sugar water. Cages contained approximately 50 adults each.

### Electroantennogram (“EAG”) Assay.

EAG recordings were obtained using a Combi Probe (Syntech, Kirchzarten, Germany) fitted with glass capillary Ag/AgCl electrodes, filled with insect Ringer solution. Fine, tapered capillaries were prepared on a Gravipull-2 capillary puller (Kation Scientific, Minneapolis, USA). Data were recorded with an IDAC-4 data acquisition system (Syntech), interfaced with a computer that had the software EAGpro (version 1.1, Syntech). Stimuli were dissolved in hexane (at 0.001%, 0.01% or 0.1% w/v) and applied to small filter papers (Whatman No. 1) (10 μL). This gave doses on the filter paper of 1 μg, 10 μg and 100 μg. After evaporation of the solvent, the filter papers were placed in a Pasteur pipette. Pipettes were attached to a CS-55 stimulus controller (Synthech) that delivered a pulse of air (30 mL/s) through the pipette and into a metal stimulus delivery tube (Ø0.95 cm×18 cm) that dispensed a continuous airflow (25 mL/s) and was positioned approximately 2 cm from the mosquito antenna so that the airflow contacted the entire antenna. The duration of the pulse was 0.7 s.

Screening took place on A. gambiae females 4-7 days post-eclosion. Females were starved (sugar water replaced with distilled water in the colony cage) for 24 hours before trials to ensure a standard hunger level in test subjects. Mosquitoes were prepared for trials in this manner: the head of the mosquito was excised and the base of the head was connected to the ground electrode. The tip of the distal segment of one antenna was cut and inserted it into the recording electrode. Only mosquitoes producing a steady baseline reading were used for trials. All readings were taken within 10 minutes of the time the mosquito was prepared and each female was tested only once with one full chemical series.

### Stimuli and Order of Stimulation.

Trials included human scent emanating from a sock (Tradition brand, nylon) previously conditioned through 30 minutes of vigorous exercise by a volunteer and incubated at 28° C. and 80% RH for 24 hours. Odors in human sweat have previously been found to be attractive to A. gambiae. For trials, the sock was cut into 0.5 cm×3 cm strips and put into the pipette dispensing the odor pulses.

Referring to FIG. 1, seven diethers (3a{3,4} (1-butoxy-2-propoxybenzene), 3a{3,6} (1-allyloxy-2-propoxybenzene), 3b{3,6} (1-allyloxy-3-propoxybenzene), 3c{1,3} (1-methoxy-4-propoxybenzene), 3c{2,2} (1,4-diethoxybenzene), 3c{3,6} (1-allyloxy-4-propoxybenzene), cy{3,3} (5-(2′-propoxyethyl)-1-propoxycyclopent-2-ene)) were screened along with DEET™ (used as a positive control). The compounds numbered 3 were synthesized as individual congeners of dialkoxybenzene libraries used in previous studies with the gypsy moth and the cabbage looper (FIG. 1). Racemic compound cy{3,3} was a congener of a series of diethers of cis 5-(2′hydroxyethyl)-cycloprop-2-en-1-ol, tested for their detection by male gypsy moths.

Odors in each run were presented to the mosquito in the following order with at least 25 seconds between odor pulses: no odor, no odor, 10 μg DEET™, 10 μg chemical, no odor, no odor, human scent only, human scent with 1 μg chemical, human scent with 10 μg chemical, human scent with 100 μg chemical, human scent only, no odor, no odor, 10 μg DEET™. Several of these pulses served as internal controls to ensure that the mosquito antenna was still responding to the stimuli, or the human odor was detectable to the mosquito. One of the pulses, such as air pulse, produced a response, but it is smaller than that of odor or of presented compounds, seven diethers. The DEET™ pulses were included as a reference. The pulses of interest were those containing both the human odor and chemical of interest in various concentrations. 9 runs were completed for each chemical tested and recorded the amplitude of the peaks produced for each odor pulse. The activity of each treatment was computed by comparing the amplitude of the peak of interest to the DEET™ reference peak. This was done to standardize measurements between runs.

### Behavioural Assay.

Olfactometer Apparatus.

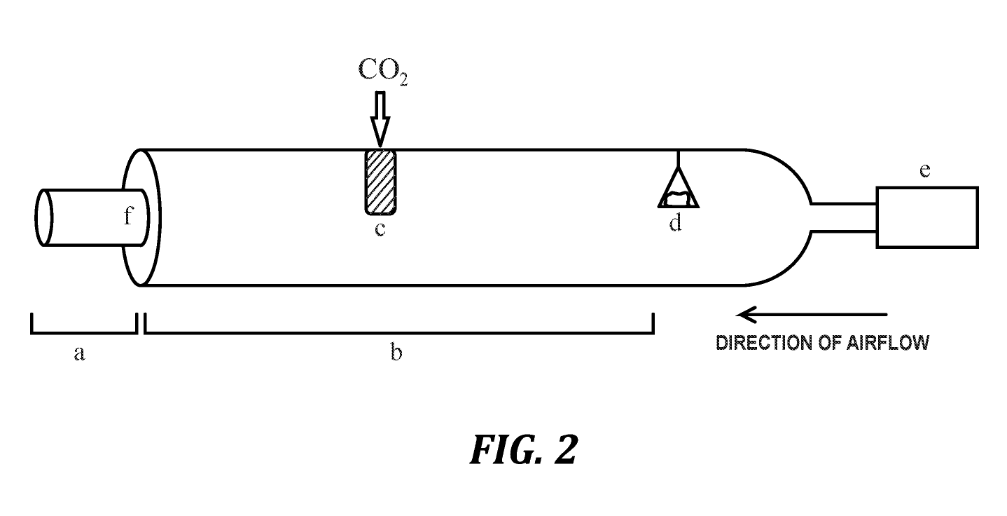

The olfactometer procedure and materials used in this study are similar to those employed by Zappia and Roitberg (2012) Energy-state dependent responses of Anopheles gambiae (Diptera: Culicidae) to simulated bednet-protected hosts. J. Vector Ecol. 37 (1): 173-8, herein incorporated by reference in its entirety. The behavioral assay was conducted in a custom-made glass olfactometer tube (145 cm long, 17 cm diameter) (FIG. 2). A mosquito release chamber was located on one side of the tube and consisted of a small tube with a gate attached separating the mosquito from the experiment arena. A small sac (10 cm×3 cm in diameter) made of mesh (Onsight equipment insecticide free hiker's mosquito shelter) was positioned 60 cm from the end of the release chamber. The mesh sac was immersed in the treatment compound immediately before trials and allowed to dry before the trial began.

The sac contained human odor in the form of a sock (Secret brand, nylon) conditioned through 30 minutes of strenuous exercise and incubated at 28° C. and 80% RH for 24 hours before use. This sock was tested to ensure it was attractive to mosquitoes at the beginning of each trial day (by placing it in a non-experimental colony cage and observing whether females in the cage would seek it out to probe at it) and replaced every 5 days. A breathing tube was also attached to the human odor/chemical complex that allowed a one-way flow of exhaled carbon dioxide into the arena through the sock to make the human odor more attractive to A. gambiae. Carbon dioxide was exhaled into this tube once every 15 seconds during trials.

An odor-producing sugar source was located 73 cm from the human odor/chemical complex. This consisted of a cotton ball impregnated with approximately 5 g of honey (Kidd Bros, Alfalfa Clover). The ball was suspended on a metal stage from the top of the olfactometer tube. This gave the mosquito an alternative during trials from seeking the human odor source. At the opposite end of the tube from the release gate was an air source pumping a one-way source of carbon-filtered air into the olfactometer arena flowing from the sugar source to the human odor to the release gate at a rate of 0.025 L/s. The olfactometer was rinsed daily with acetone and water to minimize residual odors.

Assay Procedure.

A 2×5 factorial design was used with solvent type and chemical as factors. The solvent types tested were a 1:1 and 1:5 ratio of isopropyl alcohol to water. Two different solvent types were assessed to determine the lowest ratio of alcohol to water that would be effective, since a lower concentration of alcohol is both more cost effective and practical in the field. EAG screening indicated that 3c{3,6} would be an appropriate chemical for further study. In addition, we tested DEET™ as a positive control compound. Two concentrations of DEET™ and 3c{3,6} (0.1% and 1%) and a control were assessed for each solvent consisting of the solvent without the active ingredient (0% concentration).

Female A. gambiae (4-7 days old, not previously blood-fed, and without a visibly swollen abdomen, wing size 2.93 mm-3.49 mm) were starved for 24 hours before assays. All assays were run under dim red light (1.79 μmol photons/(s×m²)) in the colony chamber 1-5 hours after darkness since this is the period in which blood-seeking behaviour in females is highest. Mosquitoes were allowed to acclimate in the release chamber for 3 minutes before trials began. After 3 minutes, the release gate was opened. If the mosquito did not leave the release chamber within 3 minutes of when the gate was opened the release tube was given a gentle tap and if the mosquito failed to leave within 9 minutes from when the release gate was opened she was considered to be non-responsive and was not included in the trials. When the mosquito entered the arena, timing began.

During trials, the olfactometer design was such that the mosquito encountered the human odor/chemical first, followed by the sugar odor source. The experiment arena consisted of the area in the olfactometer that was placed in front of the plant odor source, relative to the release point of the mosquito. Trials ended when the mosquito landed anywhere in the arena and rested for 5 minutes or flew to the plant sugar source. The trial was terminated if the mosquito flew to the sugar source as it was considered to have abandoned the human odor source in favour of the sugar source. The trial was also ended if the mosquito was still active after 30 minutes and had not generated any of the termination criteria. The manner by which the trial ended was recorded. Additionally, the total experiment time, the amount of time the mosquito spent on the chemical/human odor complex (probe time) and the number of times the mosquito attempted to probe the chemical/human odor source were measured. Mosquitoes were killed and dried immediately following trials and wing-length was measured by photographing the wing and measuring it with the software Analyzing Digital Images (Version 12.0.1). This was done to ensure females in different treatments were of a comparable size.

Data Analysis.

All data were analyzed in R version 2.15.0 (R Development Core Team, 2012). In order to assess whether the EAG set-up was functional, an air pulse containing no odor was compared to the DEET reference pulse using a paired T-test to ensure that females were responding to the odors presented and not the air pulse. EAG activity values were analyzed using a generalized least squares ANOVA with a variance structure that accounts for measurements within runs being more similar than measurements between runs. The chemical and the concentration of the chemical tested were used as factors in this analysis. Behavioural assay data was analyzed using a MANOVA with solvent type and chemical (including concentration) as factors. Total experiment time, probe time and number of attempted probes at the human odor source were grouped in analyses as these were expected to be related within trials. Post-hoc Tukey's HSD tests were conducted on each of the response variables separately to determine which treatments differed from each other. Whether the test ended by the mosquito flying to the honey source or resting for 5 minutes was analyzed with a binomial generalized linear model with chemical and solvent type as factors.

### Results

EAG Screening.

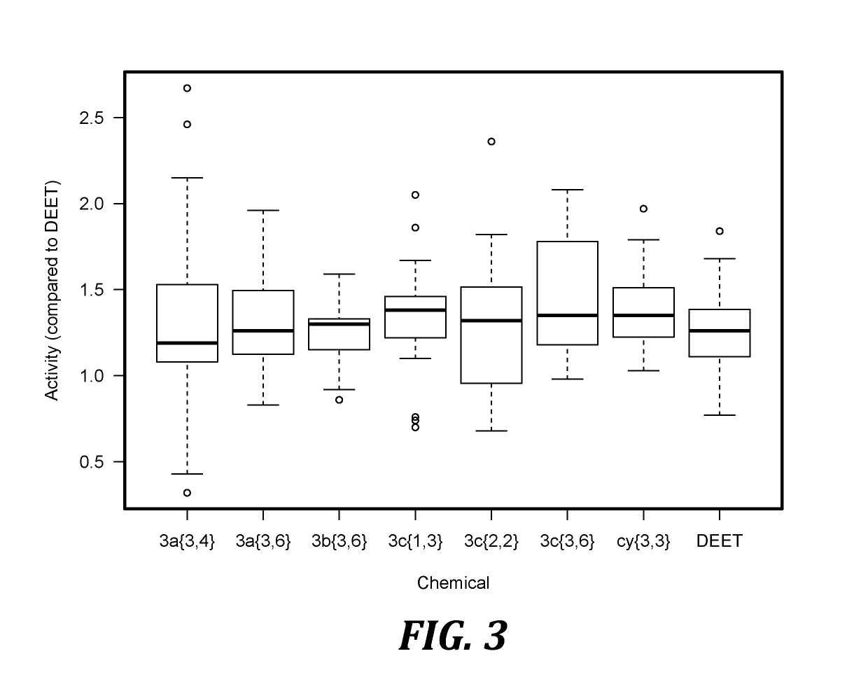

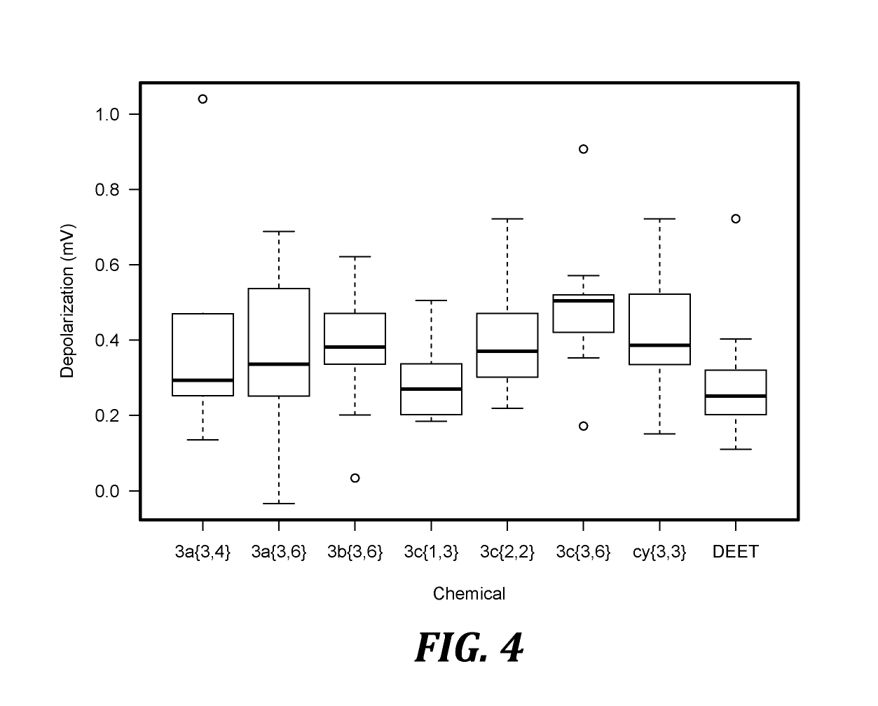

All tested mosquitoes responded to the human odor and to DEET (positive control) indicating that the EAG set up was functional. Response to the air pulse was 51% less than for the DEET reference pulse indicating that A. gambiae responded to the chemical stimuli (t=−13.13, df=71, P<0.001). The antennae responded to all compounds by themselves, and no difference was found in activity across different chemicals or concentrations tested (gls: df=219, P>0.05 for all comparisons) (FIG. 3). However, there was also no difference in comparison with DEET™ indicating that all chemicals tested produced a comparable response in the mosquito to DEET™. None of the compounds either enhanced or inhibited the antennal responses to human scent; depolarizations for the compound+scent samples were ˜50% larger than the responses to scent alone, suggesting an additive effect between the compounds or DEET™ and the human scent. 3c{3,6} was chosen for the behavioural assay since this chemical had the largest activity of all the compounds tested. In addition, this chemical alone (without the human odor) also produced a larger air-corrected antennal depolarization than the other compounds tested (FIG. 4). However, this difference was not significant (gls: df=73, P>0.05).

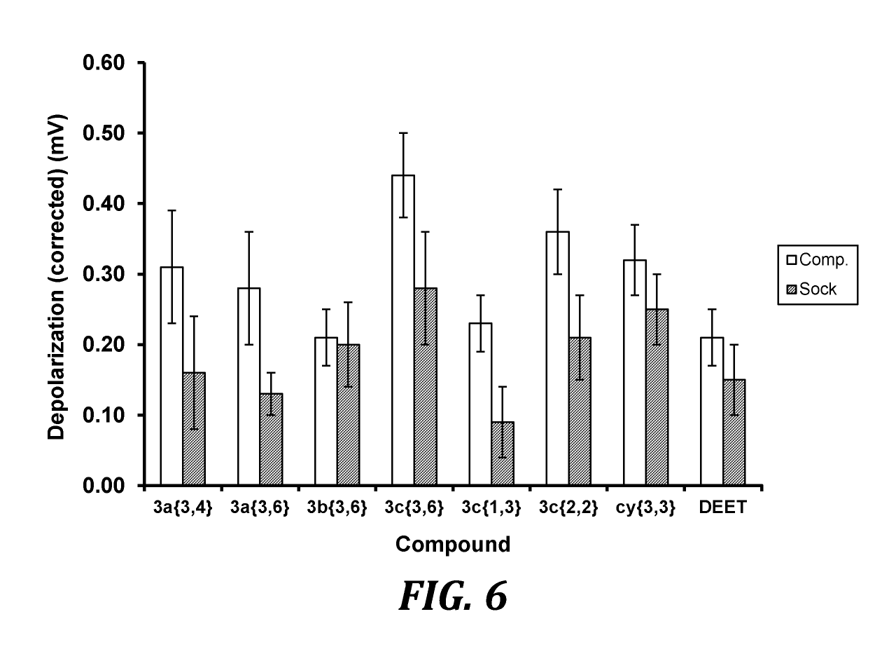

As discussed above, the compound (3c{3,6}) provided one of the largest depolarization responses by itself: larger than 3b{3,6} (the meta isomer of 3c{3,6}) or 3c{1,3} (FIG. 6). Furthermore, the compounds by themselves provide depolarizations comparable to the respective “stocking” (or “sock”) stimuli. All the tested compounds provided responses in the mixed treatments that are larger than each (sock or compound) by themselves, but smaller than the sum of (sock+compound), suggesting that there is interaction or saturation between the compounds.

When comparing the first and second stocking stimuli, none of the compounds significantly decrease the depolarization caused by the second stocking stimulus, suggesting that the antenna is not getting exhausted or adapted and also that the compounds and the blended stimuli had no long-term effect.

### Behavioural Assays.

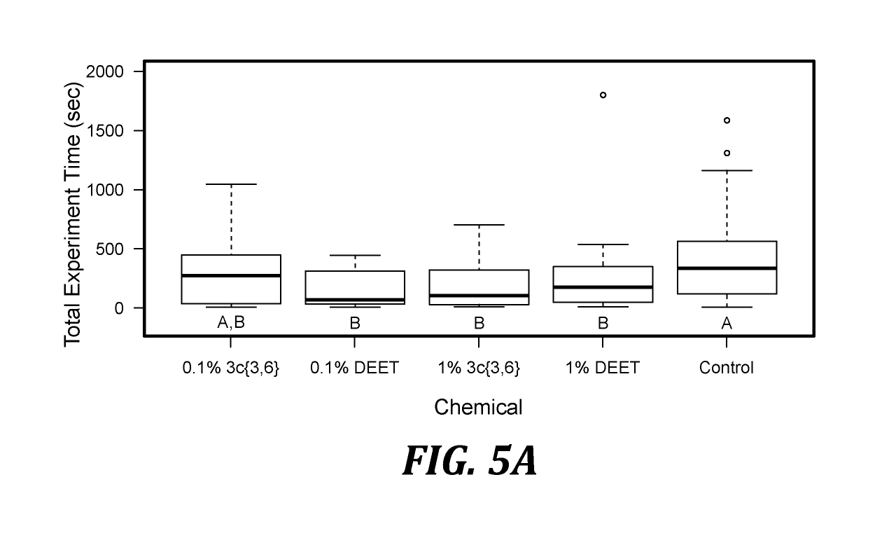

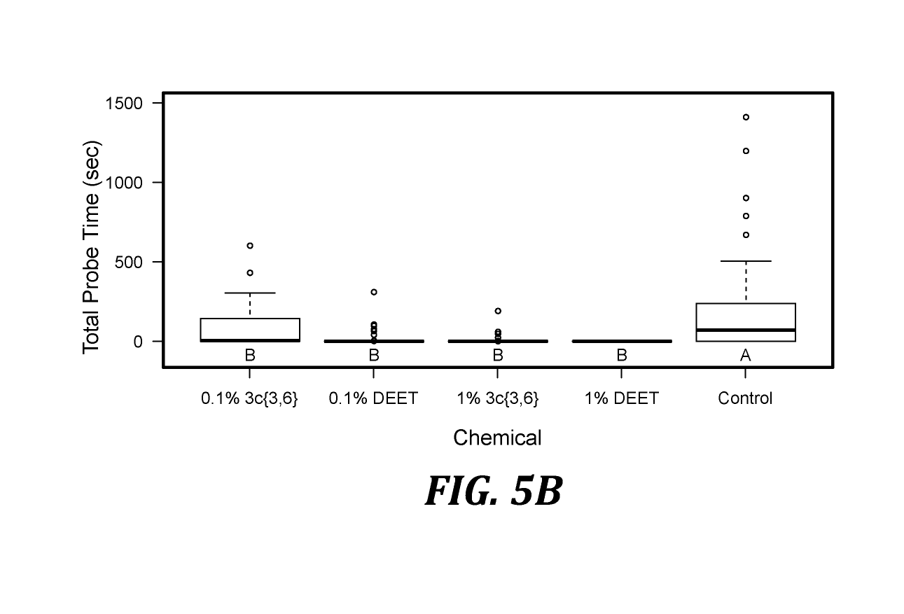

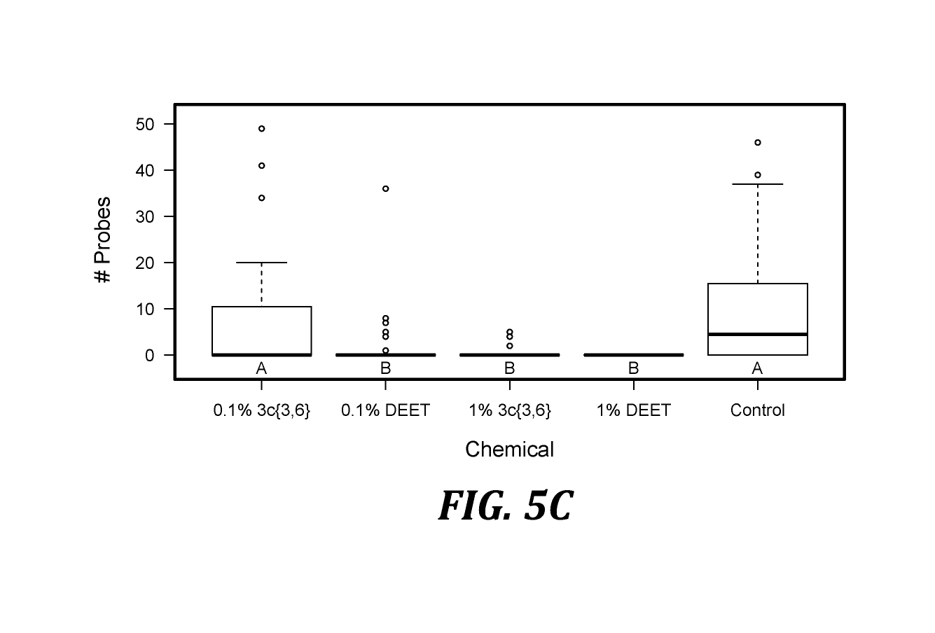

The chemical A. gambiae was exposed to affected total trial time, probe time and number of probe attempts at the human odor source (MANOVA: F=5.233, df=4, P<0.001) (FIGS. 5A-5C), however, solvent type did not have an effect on A. gambiae behaviour (MANOVA: F=2.081, df=1, P=0.104). Tukey's HSD analyses revealed that A. gambiae response differed significantly from the unprotected control condition when a human odor source was protected with 1% DEET™ or 1% 3c{3,6} (P<0.05 for total experiment time, total probe time and total number of probes). These analyses found that A. gambiae individuals spent less time in the proximity of the human odor source, spent less time on the human odor source, and attempted to probe less when a human odor was protected with one of these chemicals (FIGS. 5A-5C). Whether the trial ended by the mosquito flying to the sugar odor source or resting was not affected by either chemical or solvent type (GLM: df=199, P>0.05 for all chemical and solvent comparisons).

### DISCUSSION

Control of anopheline mosquito species is a cornerstone of malaria eradication programs (WHO 2012). Due to changes in vector behavior over time and in response to current control strategies (for example, development of insecticide resistance) new vector control strategies are necessary in order to limit the impact of malaria and meet malaria targets. Repellents are widely used to deter arthropod attacks on humans. The efficacy of a new repellent candidate 3c{3,6} was evaluated through two assays. The physiological response of A. gambiae mosquitoes was investigated in response to this chemical and assessed how A. gambiae mosquitoes would respond when presented with a simulated host protected with this chemical and an alternate food source. The results of these assays indicate that 3c{3,6} is an effective repellent for A. gambiae, with both the physiological and behavioural response produced in these assays being comparable to that of DEET.

DEET elicited olfactory responses by itself in the EAG screens. Furthermore, DEET did not affect the responses to the human scent, neither short-term by inhibition or enhancement nor long-term by inhibition of the responses to human scent. The diethers tested all elicited olfactory responses similar to DEET and also did not modulate the responses of the A. gambiae antennae to human scent.

Behavioural assays indicate that 3c{3,6} is an effective repellent against A. gambiae. Mosquitoes presented with a human odor protected with this chemical spent less time in the proximity of a human odor and did not attempt to gain a blood meal as often as mosquitoes presented with an unprotected human odor. A 1% solution of 3c{3,6} was found to produce a comparable response in mosquitoes to a 1% solution of DEET™; however, a 0.1% solution of DEET™ was more effective at repelling A. gambiae individuals than a 0.1% solution of 3c{3,6} (FIGS. 5A-5C), indicating that at least a 1% solution should be used for further experimentation.

The chemical the host was protected with did not affect trial termination method. The aim of measuring the method in which the trial terminated was to test whether A. gambiae would persist in the proximity of a human odor source that was protected with a repellent (i.e. stay in the experiment arena) or pursue another available food source (i.e. fly to the honey). A shift towards leaving the experiment arena in favour of the sugar food source would indicate a reduced risk of future attack for the simulated host in this experiment. There are several reasons that an increased rate of seeking out an alternate food source was not found in response to a simulated host protected with a repellent.

First, the experimental arena may not have approximated natural conditions well enough to detect this response, if present. This may have been due to the experimental set-up being small scale compared to natural conditions or that the alternate food source was not attractive enough (although we have done preliminary tests suggesting this is not the case). It is more likely, however, that the expected result was not detected due the feeding behaviour of A. gambiae. It is predicted that A. gambiae will seek out a blood meal whenever possible unless it has just taken a nectar meal or host densities are high. Therefore, it is possible that, even in the case that the host is protected by a repellent, the mosquito will still seek a blood meal when available as a blood meal is of superior quality compared to a nectar meal in terms of fitness gain. This is predicted even when the blood host is protected with a ITN, although it was also found in this study that an ITN can make mosquito emigration from a human dwelling more likely if the mosquito estimates the toxin level present to be high.

Developing successful malaria control strategies is an ongoing challenge. Vector control is a target of malaria control programs, but is made difficult due to changes in vector behaviour due to control strategies and an imperfect knowledge of vector behaviour. The use of repellents in addition to current control strategies has been advocated, however, concern over the safety of DEET™, the most commonly used repellent, has created a need for new, safer repellents. This study examined mosquito behaviour in response to a potential repellent 1-allyloxy-4-propoxybenzene, 3c{3,6}. 3c{3,6} was found to be an effective repellent for A. gambiae, causing females to limit contact with a blood-host odor protected with this chemical.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for repelling an Anopheles gambiae comprising applying to a site of interest an effective amount of a compound of Formula I: 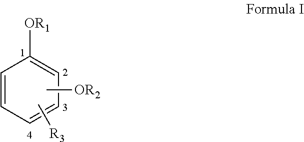
wherein R₁ is methyl, ethyl, propyl, n-butyl, or allyl;
R₂ is at positions 2, 3 or 4 and is H, methyl, ethyl, propyl, n-butyl, or allyl; and
R₃ is optionally present at positions 2, 3 and 4, and is allyl,
with the provisos that
when R₂ is at position 2, R₃ if present is at position 3, or
when R₂ is at position 3, R₃ if present is at positions 2 or 4, or
when R₂ is at position 4, R₃ if present is at position 2; and
with the proviso that the compound of Formula I does not comprise a compound according to Formula II: 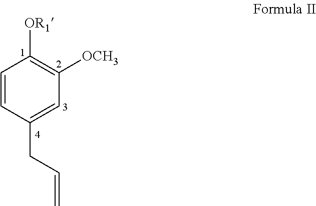
wherein R₁′ is methyl, ethyl, propyl, n-butyl, allyl, or mixture thereof;
wherein the compound of Formula I decreases the number of Anopheles gambiae attacks on a subject.

2. The method of claim 1, wherein the compound of Formula I is selected from

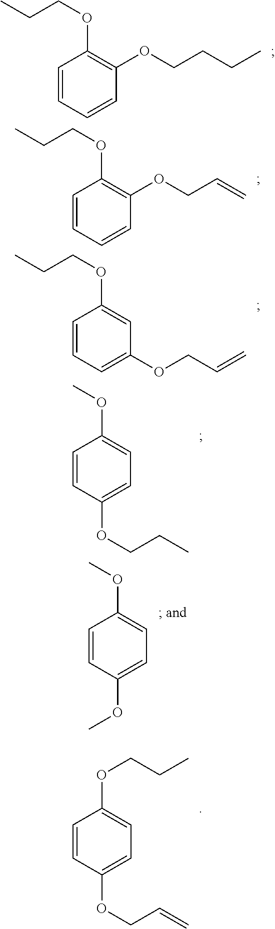

3. The method of claim 2 wherein the compound of Formula I is 1-allyloxy-4-propoxybenzene.

4. The method of claim 1, wherein the subject is a human.

5. The method of claim 1 wherein the compound of Formula I is a Anopheles gambiae repellent.

6. The method of claim 5 wherein the compound of Formula I is configured to reduce Anopheles gambiae probing at a simulated blood-host and increase a probability an Anopheles gambiae seeks a plant odor source.

7. The method of claim 1 wherein the compound of Formula I regulates a physiological olfaction response of Anopheles gambiae.

8. The method of claim 1 wherein the compound of Formula I is applied at the site of interest in an amount of from 1 μg/cm² or more to 100 μg/cm² or less.

9. The method of claim 1 wherein the site of interest is a bed net or a skin portion.

10. The method of claim 1 further comprising applying to a site of interest a composition comprising a compound of Formula I.
